(19) United States
(12) Patent Application Publication
(10) Pub. No.: US 2025/0032709 A1
(43) Pub. Date: Jan. 30, 2025

(54) ACOUSTIC FLOW SENSOR FOR CONTINUOUS MEDICATION FLOW MEASUREMENTS AND FEEDBACK CONTROL OF INFUSION

(71) Applicant: ICU Medical, Inc., San Clemente, CA (US)

(72) Inventors: Gary David SHUBINSKY, San Clemente, CA (US); Jeffrey Donald MERWIN, San Clemente, CA (US); Tamas R. BAN, San Clemente, CA (US); Ion Vergil NICOLAESCU, San Clemente, CA (US)

(21) Appl. No.: 18/790,446

(22) Filed: Jul. 31, 2024

Related U.S. Application Data
(63) Continuation of application No. 17/662,221, filed on May 5, 2022, now Pat. No. 12,076,531.
Application No. 17/662,221 is a continuation of application No. 16/308,425, filed on Dec. 7, 2018, now Pat. No. 11,324,888.
(60) Provisional application No. 62/348,301, filed on Jun. 10, 2016.

(51) Int. Cl. A61M 5/172; A61M 5/142

(52) U.S. Cl. CPC A61M 5/172; A61M 5/14212; A61M 2205/3334; A61M 2205/3375; A61M 2205/52

(57) ABSTRACT

An infusion system determines a volumetric flow rate of infusion fluid delivered by an infusion pump along a flow path based on: an upstream acoustic signal emitted by at least one upstream acoustic sensor and detected by at least one downstream acoustic sensor; a downstream acoustic signal emitted by the downstream acoustic signal and detected by the upstream acoustic sensor; and a phase delay between the upstream acoustic signal and the downstream acoustic signal either upstream or downstream. The infusion system automatically adjusts the infusion pump based on the determined volumetric flow rate to achieve a desired volumetric flow rate of the infusion fluid along the flow path.

### CROSS-REFERENCE TO RELATED APPLICATIONS

This application is a continuation of U.S. application Ser. No. 17/662,221, filed May 5, 2022, entitled ACOUSTIC FLOW SENSOR FOR CONTINUOUS MEDICATION FLOW MEASUREMENTS AND FEEDBACK CONTROL OF INFUSION, which is a continuation of U.S. application Ser. No. 16/308,425, filed Dec. 7, 2018, entitled ACOUSTIC FLOW SENSOR FOR CONTINUOUS MEDICATION FLOW MEASUREMENTS AND FEEDBACK CONTROL OF INFUSION which is a National Stage Entry of Application No. PCT/US2017/036619, filed Jun. 8, 2017, entitled ACOUSTIC FLOW SENSOR FOR CONTINUOUS MEDICATION FLOW MEASUREMENTS AND FEEDBACK CONTROL OF INFUSION, which claims benefit of U.S. Provisional Application No. 62/348,301, filed Jun. 10, 2016, entitled ACOUSTIC FLOW SENSOR FOR CONTINUOUS MEDICATION FLOW MEASUREMENTS AND FEEDBACK CONTROL OF INFUSION. The contents of the aforementioned application are hereby incorporated by reference in its entirety as if fully set forth herein. The benefit to the foregoing application is claimed under the appropriate legal bias, including, without limitation, under 35. U.S.C. § 119(e).

### BACKGROUND OF THE INVENTION

### Field of the Invention

The disclosure relates to flow detection systems and methods for operating an infusion pump which utilize a high-frequency acoustic, closed loop system to measure and control the volumetric flow rate of the infusion pump.

### Background of the Invention

Infusion systems and methods often operate in an open loop configuration, without receiving feedback regarding the volume of fluid being delivered to the patient. These infusion systems and methods typically rely on tightly controlled tolerances to fabricate the individual components and assemblies of the infusion pumping system to maintain the accuracy of delivered medication over a prescribed time. The accuracy of the amount of the prescribed medication being delivered to the patient by current infusion systems and methods can vary over time due to the component degradation over the life of the infuser. Additionally, requirements for tight tolerances for the individual components of the pumping mechanism significantly increase the manufacturing and service cost of the infusion system. Other infusion systems and methods utilize varying ways of attempting to monitor the amount of the medication being delivered to the patient with one or more additional issues.

An infusion system and method is needed to reduce one or more issues associated with one or more of the current infusion systems and methods.

### SUMMARY OF THE INVENTION

In one embodiment of the disclosure, an infusion system for automatically detecting and adjusting a volumetric flow rate delivered by an infusion pump is disclosed. The infusion system includes an infusion pump, a flow path, at least one upstream acoustic sensor, at least one downstream acoustic sensor, at least one processor, and at least one memory. The infusion pump is configured to pump infusion fluid. The infusion fluid is configured to be delivered by the infusion pump along the flow path. The upstream acoustic sensor is located at an upstream location of the flow path. The downstream acoustic sensor is located at a downstream location of the flow path. The downstream acoustic sensor is configured to detect an upstream acoustic signal emitted by the upstream acoustic sensor. The upstream acoustic sensor is configured to detect a downstream acoustic signal emitted by the downstream acoustic sensor. The processor is in electronic communication with the infusion pump, the upstream acoustic sensor, and the downstream acoustic sensor. The memory is in electronic communication with the processor. The memory includes programming code for execution by the processor. The programming code is configured to determine a volumetric flow rate of the infusion fluid along the flow path based on the upstream acoustic signal detected by the downstream acoustic sensor and on the downstream acoustic signal detected by the upstream acoustic sensor. The programming code is configured to determine the volumetric flow rate of the infusion fluid along the flow path based on a first phase delay of the upstream acoustic signal between the upstream acoustic sensor and the downstream acoustic sensor, and/or on a second phase delay of the downstream acoustic signal between the downstream acoustic sensor and the upstream acoustic sensor. The programming code is further configured to automatically adjust the infusion pump based on the determined volumetric flow rate, to achieve a desired volumetric flow rate of the infusion fluid along the flow path.

In another embodiment of the disclosure, a method for automatically detecting and adjusting a volumetric flow rate delivered by an infusion pump is disclosed. In one step, infusion fluid is delivered with an infusion pump along a flow path. In another step, an upstream acoustic signal emitted by at least one upstream acoustic sensor, located at an upstream location of the flow path, is detected with at least one downstream acoustic sensor located at a downstream location of the flow path. In an additional step, a downstream acoustic signal emitted by the downstream acoustic sensor, located at the downstream location of the flow path, is detected with the upstream acoustic sensor located at the upstream location of the flow path. In another step, a volumetric flow rate of the infusion fluid along the flow path is determined, with at least one processor, over each stroke of the infusion pump based on the upstream acoustic signal detected by the downstream acoustic sensor and on the downstream acoustic signal detected by the upstream acoustic sensor. The processor determines the volumetric flow rate of the infusion fluid along the flow path over each stroke of the infusion pump by determining a first phase delay of the upstream acoustic signal between the upstream acoustic sensor and the downstream acoustic sensor, or by determining a second phase delay of the downstream acoustic signal between the downstream acoustic sensor and the upstream acoustic sensor. In still another step, the infusion pump is automatically adjusted, with the processor, over each pumping cycle of the infusion pump based on the determined volumetric flow rate to achieve a desired volumetric flow rate of the infusion fluid along the flow path.

In still another embodiment of the disclosure, a non-transitory computer readable medium is disclosed. The non-transitory computer readable medium is configured to, using at least one processor, automatically detect and adjust a volumetric flow rate of infusion fluid delivered by an infusion pump. The non-transitory computer readable medium includes programming code to command the processor to determine, over each stroke of the infusion pump, the volumetric flow rate of the infusion fluid delivered by the infusion pump along a flow path. The programming code is configured to determine, over each stroke of the infusion pump, the volumetric flow rate based on an upstream acoustic signal emitted by at least one upstream acoustic sensor, located at an upstream location of the flow path, which is detected by at least one downstream acoustic sensor located at a downstream location of the flow path. The programming code is further configured to determine, over each stroke of the infusion pump, the volumetric flow rate based on a downstream acoustic signal emitted by the downstream acoustic sensor and detected by the upstream acoustic sensor. The programming code is configured to automatically adjust the infusion pump over each pumping cycle of the infusion pump, based on the determined volumetric flow rate, to achieve a desired volumetric flow rate of the infusion fluid along the flow path.

In certain embodiments, an infusion system can automatically control an infusion pump. The infusion system can include an infusion pump that can pump infusion fluid along a flow path. The infusion system can also include a first acoustic sensor positioned at a first location along the flow path, the first acoustic sensor can detect a first acoustic signal. The infusion system can further include a second acoustic sensor positioned at a second location downstream from the first acoustic sensor along the flow path. The second acoustic sensor can detect a second acoustic signal. The infusion system can also include a controller that can determine a first volumetric flow rate of the infusion fluid based on the detected first acoustic signal and the detected second acoustic signal. The controller can also control the infusion pump to pump infusion fluid at a second volumetric flow rate based on the detected first volumetric flow rate.

The infusion system of the preceding paragraph can have any sub-combination of the following features: wherein the first acoustic signal originated from the second acoustic sensor and the second acoustic signal originated from the first acoustic sensor; wherein the first acoustic sensor comprises a first transducer and the second acoustic sensor comprises a second transducer; wherein the first acoustic sensor comprises a first transmitter and a first receiver and the second acoustic sensor comprises a second transmitter and a second receiver; wherein the first receiver and the second receiver each comprise at least one noise cancelling component; wherein the first volumetric flow rate of the infusion fluid is calculated over each stroke of the infusion pump; wherein the first volumetric flow rate is determined based on a first phase delay associated the first acoustic signal; wherein the first volumetric flow rate is determined based on a second phase delay associated the second acoustic signal; wherein the first volumetric flow rate is determined based on a length between the first location and the second location; wherein the first volumetric flow rate is determined based on a first time it takes the first acoustic signal to travel between the second acoustic sensor and the first acoustic sensor; wherein the first volumetric flow rate is determined based on a first time it takes the second acoustic signal to travel between the first acoustic sensor and the second acoustic sensor.

In certain embodiments, a method of controlling an infusion pump can include detecting a first acoustic signal from a first acoustic sensor positioned at a first location along the flow path. The method can further include detecting a second acoustic signal from a second acoustic sensor positioned at a second location downstream from the first acoustic sensor along the flow path. The method can also include determining a first volumetric flow rate of the infusion fluid based on the detected first acoustic signal and the detected second acoustic signal. Moreover, the method can include changing the first volumetric flow rate to a second volumetric flow rate based on the determined first volumetric flow rate.

The method of the preceding paragraph can have any sub-combination of the following features: wherein the first acoustic signal originated from the second acoustic sensor and the second acoustic signal originated from the first acoustic sensor; wherein the first acoustic sensor comprises a first transducer and the second acoustic sensor comprises a second transducer; wherein the first acoustic sensor comprises a first transmitter and a first receiver and the second acoustic sensor comprises a second transmitter and a second receiver; wherein the first receiver and the second receiver each comprise at least one noise cancelling component; wherein the first volumetric flow rate of the infusion fluid is calculated over each stroke of the infusion pump; wherein the first volumetric flow rate is determined based on a first phase delay associated the first acoustic signal; wherein the first volumetric flow rate is determined based on a second phase delay associated the second acoustic signal; wherein the first volumetric flow rate is determined based on a length between the first location and the second location; wherein the first volumetric flow rate is determined based on a first time it takes the first acoustic signal to travel between the second acoustic sensor and the first acoustic sensor; wherein the first volumetric flow rate is determined based on a first time it takes the second acoustic signal to travel between the first acoustic sensor and the second acoustic sensor.

These and other features, aspects and advantages of the disclosure will become better understood with reference to the following drawings, description and claims.

### BRIEF DESCRIPTION OF THE DRAWINGS

FIG. 1 illustrates one embodiment of a box diagram of an infusion system;

FIG. 1A illustrates one embodiment of a pictorial view of a front of an infusion pump with a pump cassette or infusion set cassette inserted into the infusion pump;

FIG. 2 illustrates one embodiment of a side-view of an infusion set cassette illustrating configuration and placement of ultrasonic acoustic transducers;

FIG. 2A illustrates another embodiment of a side view of an infusion set cassette showing configuration and placement of ultrasonic acoustic transducers;

FIGS. 2B-2D illustrate various embodiments of a partial cross-sectional view of a flow path of an infusion system showing the configuration and placement of ultrasonic acoustic transducers;

FIG. 3 illustrates one embodiment of a simplified circuit diagram showing an acoustic flow sensing system;

FIG. 3A illustrates another embodiment of a simplified circuit diagram showing an acoustic flow sensing system;

FIG. 3B illustrates still another embodiment of a simplified circuit diagram showing an acoustic flow sensing system;

FIG. 4 illustrates one embodiment of a graph showing two acoustic pressure function curves, with time plotted on the X-axis and pressure plotted on the Y-axis, and a phase delay (difference between the received signal and the emitted signal) between the pressure function curves;

FIG. 5 illustrates one embodiment of a set of graphs in which two different frequencies are transmitted, received, and processed by two different transducers respectively;

FIG. 6 illustrates one embodiment of a flowchart showing a method of automatically detecting and adjusting a volumetric flow rate delivered by an infusion pump;

FIG. 6A illustrates another embodiment of a flowchart showing a method of automatically detecting and adjusting a volumetric flow rate delivered by an infusion pump; and

FIG. 7 illustrates one embodiment of a graph, with time plotted on the X-axis and flow velocity plotted on the Y-axis, showing a flow profile when pumping is done by intermittent movement of a pumping mechanism creating a periodic flow profile.

### DETAILED DESCRIPTION

The following detailed disclosure describes one or more modes of carrying out the invention. The disclosure is not to be taken in a limiting sense, but is made merely for the purpose of illustrating the general principles of the disclosure, since the scope of the disclosure is best defined by the appended claims. It is noted that the figures are purely for illustrative purposes and are not to scale. It is further noted that any portions of the embodiments of the below disclosure may be, in varying embodiments, combined in part or in full, one or more components may be added, or one or more components may be removed.

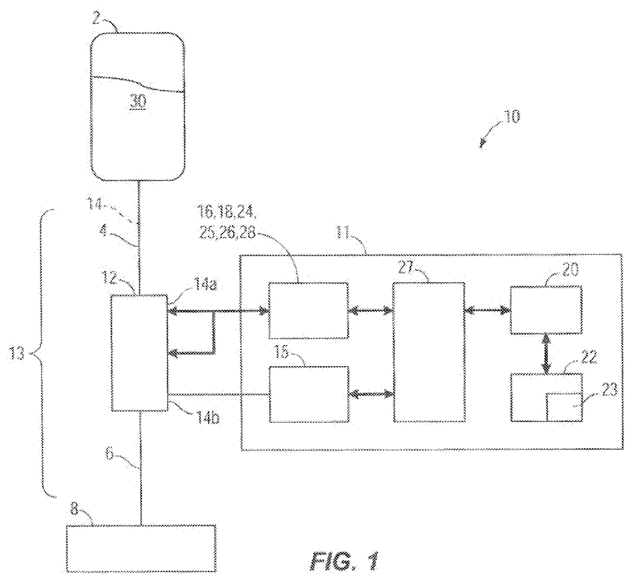

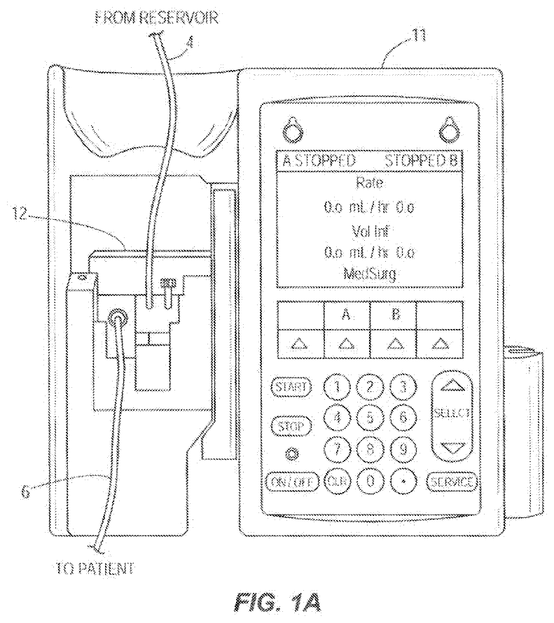

FIG. 1 illustrates an embodiment of a box diagram of an infusion system 10. The infusion system 10 can include an infusion pump or infuser 11 and an infusion set 13 that is inserted in, and is acted upon by the infusion pump 11. The infusion set 13 can include, among other elements, an inlet tube 4, an outlet tube 6 and a cassette 12. The infusion system 10 can also include an infusion pump 11 with an infusion pumping mechanism 15, a pump cassette 12, an infusion set 13 that has an internal flow path 14, at least one upstream acoustic sensor 16, at least one downstream acoustic sensor 18, at least one hardware processor 20, at least one memory (also referred to herein as a non-transitory computer readable medium) 22, programming code 23, a proximal air-in-line sensor 24, a proximal pressure sensor 25, a distal pressure sensor 26, and a distal air-in-line air sensor 28. Signals from the infusion pumping mechanism 15 and the sensors 16, 18, 24, 25, 26, 28 are acquired, conditioned if necessary, and sent to the hardware processor 20 by signal acquisition electronics 27 to monitor proper operation of fluid delivery. The hardware processor 20 can be programmed to execute programming code 23 or various algorithms stored in the memory 22 and can control operation of the pumping mechanism 15 through the driving electronics. The programming code or instructions can be implemented in C, C++, JAVA, or any other suitable programming languages. In some embodiments, some or all of the portions of the programmed instructions can be implemented in application specific circuitry 928 such as ASICs and FPGAs.

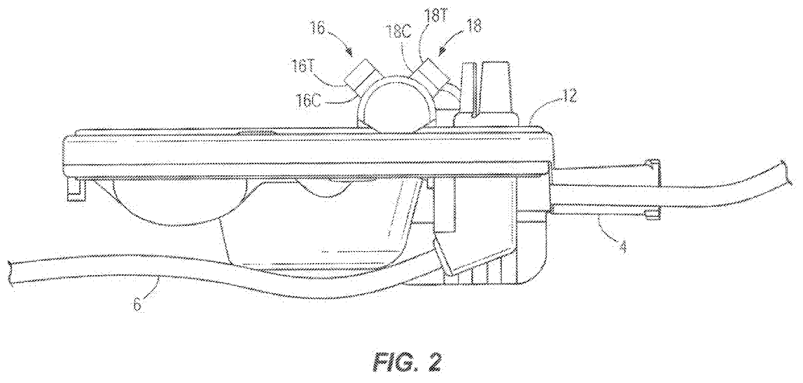

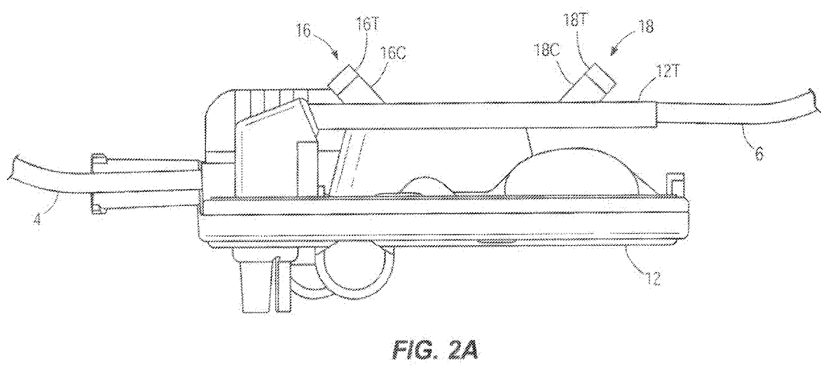

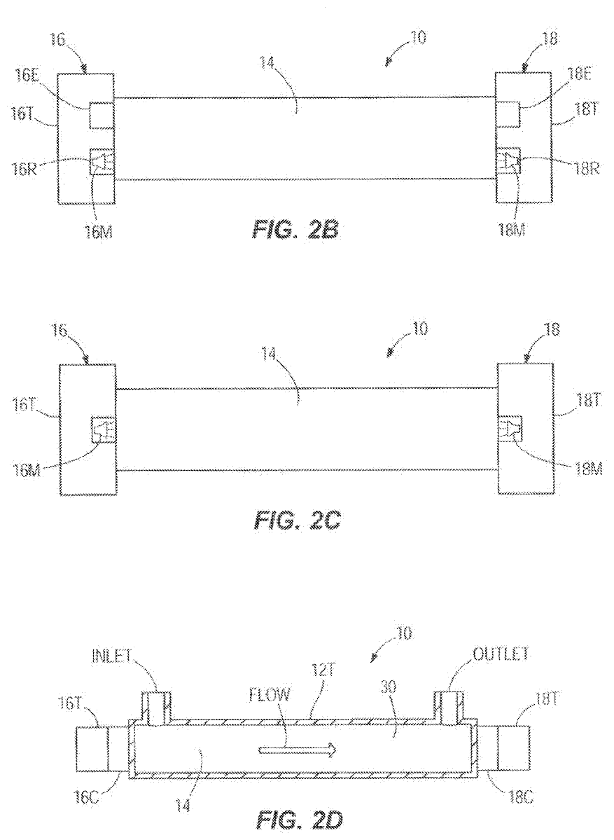

The infusion system 10 is configured to automatically detect and adjust a volumetric flow rate of infusion fluid 30 delivered by the infusion pump 11 along the flow path 14. In other embodiments, the infusion system 10 may include varying components varying in number, size, type, orientation, configuration, location, or function. For instance, in another embodiment the infusion system 10 may not utilize a pump cassette 12 (for example, see FIG. 2B and the description below).

As shown collectively in FIGS. 1 and 1A, the infusion pump 11 is operatively coupled to the pump cassette 12. In other words, the cassette 12 is inserted in the infusion pump 11. The infusion pump 11 is configured to operate on the cassette 12 to pump the infusion fluid 30 from a source or reservoir 2, which can be a bag, vial or other container, through the inlet tube 4, at least one internal passageway of the cassette 12, and through the outlet tube 6 along the flow path 14, which may lead to a patient 8. In other embodiments, the flow path 14 may be a flow path outside the pump cassette 12 or the pump cassette 12 may not be present at all and the flow path 14 may be any flow path over which the infusion fluid 30 flows. The upstream acoustic sensor 16 is coupled with the pump cassette 12 and located at an upstream location 14a of the flow path 14 at or integrated with the proximal pressure sensor 25 which is coupled with the pump cassette 12. In other embodiments, the upstream acoustic sensor 16 may be located at or integrated with the proximal air-inline sensor 24 or located or integrated with varying components of the infusion system 10. The upstream acoustic sensor 16 may include one or more ultrasonic sensors or other types of acoustic sensor. Upstream and downstream are relative terms used herein to indicate the position of the sensors along the flow path. In an embodiment, the upstream sensor encounters the flow of fluid from the reservoir 2 before the downstream sensor.

In an embodiment, the downstream acoustic sensor 18 is coupled with the pump cassette 12 and located at a downstream location 14b of the flow path 14 at or integrated with the distal air-in-line sensor 28 which is coupled with the pump cassette 12. In other embodiments, the downstream acoustic sensor 18 may be located at or integrated with the distal pressure sensor 26 or located or integrated with varying components of the infusion system 10.

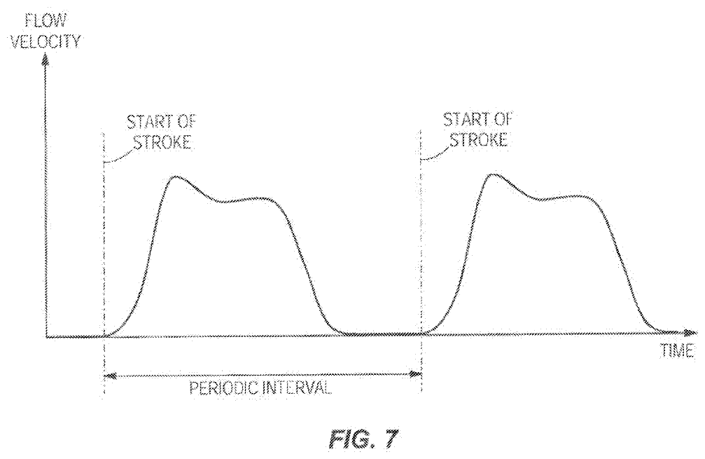

The downstream acoustic sensor 18 may include one or more ultrasonic sensors or other types of acoustic sensors. In an embodiment, the downstream acoustic sensor 18 is configured to detect an upstream acoustic signal emitted by the upstream acoustic sensor 16. The upstream acoustic sensor 16 can also be configured to detect a downstream acoustic signal emitted by the downstream acoustic sensor 18. In one embodiment, the upstream acoustic sensor 16 and the downstream acoustic sensor 18 may take turns, synchronized with the pumping mechanism of the infusion pump 11, transmitting their respective upstream acoustic signal and downstream acoustic signal. The hardware processor 20 can calculate the flow rate of the infusion fluid by integrating the phase delay measurement between the upstream and downstream signals over each periodic pumping interval. The pumping mechanism 15 operates in a periodic manner, the flow measurement is obtained by the sensors 16 and 18, and the flow is calculated by integration of the phase delay measurement over each individual periodic pumping interval as seen in FIG. 7. The flow measurement obtained by the sensors 16 and 18 can be synchronized with the period interval of the pumping mechanism 15 by the hardware processor 20 which determines the phase delay between the upstream and downstream signals over each stroke of the pumping mechanism 15 (shown in FIG. 1) and uses the phase delay to determine the volumetric flow rate of the infusion fluid over each stroke of the pumping mechanism 15. Moreover, the hardware processor 20 (shown in FIG. 1) is configured to make flow rate adjustments to the infusion fluid over each pumping cycle based on the determined flow rate over each stroke of the pumping mechanism 15. Furthermore, the hardware processor 20 (shown in FIG. 1) may average the determined flow rates over a multitude of pump cycles and adjust the flow rate of the infusion fluid accordingly. In such manner, rapid update rate fluid delivery pumping correction can be achieved utilizing periodic sampling over the pumping period.

It is noted that the movement of infusion fluid into an IV line is modulated by numerous items. The items that modulate the fluid flow are complex including but not limited to plunger movement, pump valve operation, bag height, and patient relation to the pump chamber. Many of the dynamic changes are periodic in nature as depicted in FIG. 7. This periodicity can be utilized to create a more rapid update rate for average flow. To have a reading of average flow by conventional means it may require averaging the readings over several periodic periods. However, by gating the averaging by the periodic period the average can be known in one periodic period. This can allow the control of fluid delivery to be corrected much more quickly. In some embodiments, this is much more accurate for determining the flow rate than conventional methods. In such manner, the hardware processor of the infusion system can calculate the phase delay over each stroke to determine the flow rate over each stroke and by subsequently adjusting the flow rate over each pumping cycle based on the determined flow rate, the infusion system has superior flow rate accuracy over conventional systems and methods. This allows for the use of the infusion system over a much longer period of time as diaphragm deterioration and other deterioration of the components of the infusion system may be accounted for in real-time during each pumping cycle of the pumping mechanism by adjusting the flow rate of the infusion fluid accordingly.

In another embodiment, the upstream acoustic sensor 16 and the downstream acoustic sensor 18 may continuously transmit their respective upstream acoustic signal and downstream acoustic signal or the measurements may occur over a given number of periodic intervals. One skilled in the art will recognize that the terms “upstream” and “downstream”, as used herein, are terms that describe the location of one component of the system with respect to one or more other components of the system. The flow path 14 can be thought of as a river where fluid is normally flowing from the source 2 to the patient 8. In an embodiment, the upstream acoustic sensor 16 is located between the pumping mechanism 15 and the reservoir 2 or upstream of the pumping mechanism 15, and the downstream acoustic sensor 18 is located between the pumping mechanism 15 and the patient 8 or downstream of the pumping mechanism 15. The upstream acoustic sensor 16 is located upstream along the normal flow path 14 from the downstream acoustic sensor 18. One skilled in the art will also appreciate that the upstream and downstream acoustic sensors 16 and 18 could be referred to as proximal and distal acoustic sensors respectively.

FIGS. 2 and 2A illustrate two different possible configurations and placements of acoustic sensors 16 and 18 against or adjacent to a cassette 12 as shown relative to the inlet tube 4 and the outlet tube 6. The acoustic sensors 16 and 18 are non-invasive ultrasonic transducers 16T and 18T. The transducers 16T and 18T have double function of emitters and receivers. The transducers 16T and 18T contact the cassette 12 by means of coupling elements. The coupling elements 16C and 18C are made of a compliant material and ensure good acoustic contact. In one embodiment, the transducers 16T and 18T with their coupling elements 16C and 18C are part of the pump 11 (shown in FIG. 1).

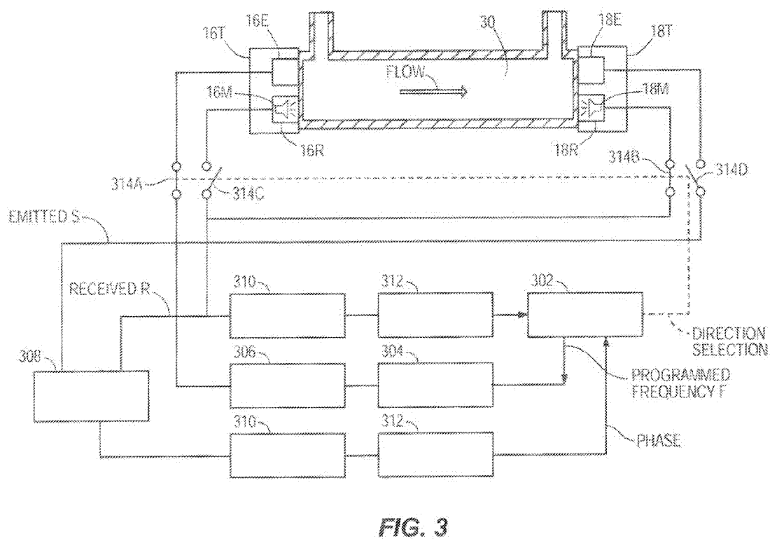

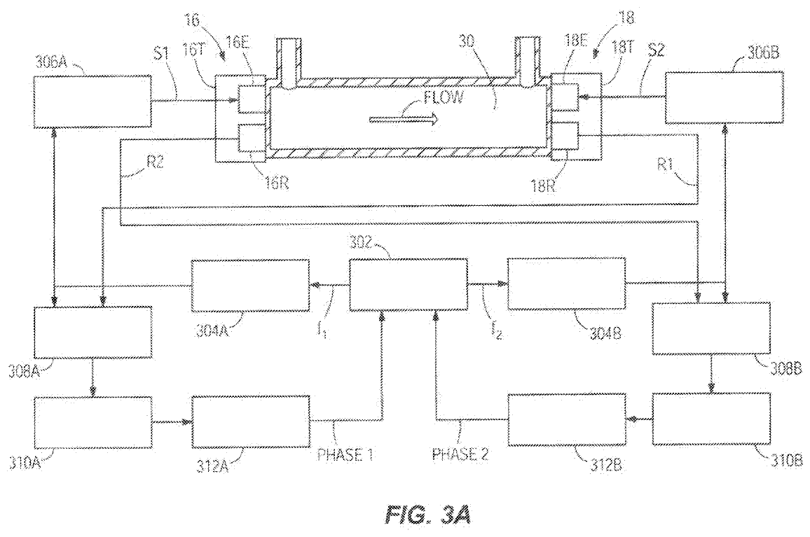

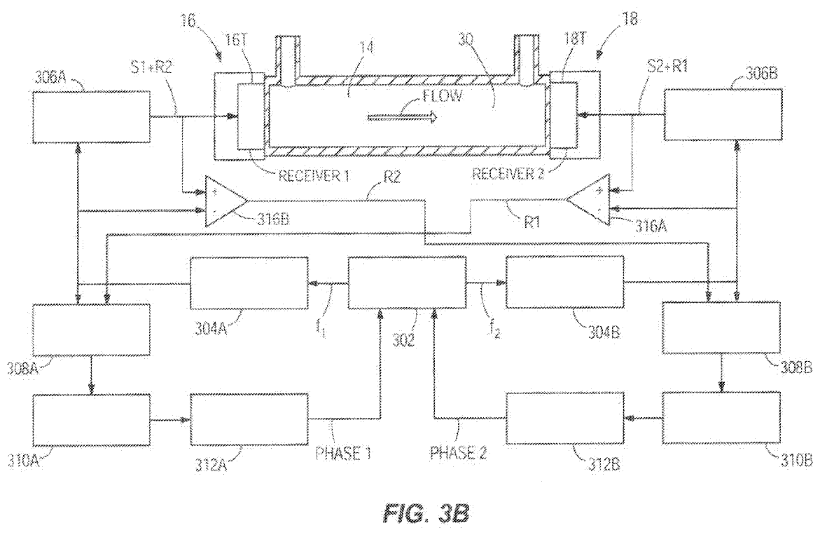

FIG. 2B illustrates one embodiment of a cross-section view through a portion of an infusion system 10 in which the disclosure could be implemented showing the flow path 14, the one upstream acoustic sensor 16, and the one downstream acoustic sensor 18. The upstream acoustic sensor 16 includes a first transducer 16T that includes a transmitter 16E and a first receiver 16R which are shown in greater detail with their accompanying electronics in FIG. 3. The first receiver 16R includes a noise cancelling component such as a noise cancelling microphone 16M. Similarly, the downstream acoustic sensor 18 includes a second transducer 18T that includes a transmitter 18E and a second receiver 18R which are shown in greater detail with their accompanying electronics in FIG. 3. The second receiver 18R can also include a noise cancelling component such as a noise cancelling microphone 18M. In additional embodiments, the upstream acoustic sensor 16 and the downstream acoustic sensor 18 may further vary.

FIG. 2C illustrates another embodiment of a cross-section view through a portion of an infusion system 10 in which the disclosure could be implemented showing the flow path 14, the upstream acoustic sensor 16, and the downstream acoustic sensor 18. The upstream acoustic sensor 16 can include a first transducer 16T, and the downstream acoustic sensor 18 includes a second transducer 18T. The first transducer 16T and the second transducer 18T may each include or be connected to a noise cancelling component such as noise cancelling microphones 16M and 18M. In additional embodiments, the upstream acoustic sensor 16 and the downstream acoustic sensor 18 may further vary.

In another embodiment shown in FIG. 2A, the existing cassette 12 is enhanced by the addition of a straight rigid tube 12T with a precise internal bore. The tube 12T can be part of the cassette 12 as an extension of its outlet port. The transducers 16T and 18T make contact with the tube 12T or are coupled to the tube 12T by the coupling elements 16C and 18C.

In another embodiment shown in FIG. 2D, the infusion system 10 may or may not include a cassette, but includes in the flow path 14 a straight rigid tube 12T that has a precisely dimensioned internal bore. If the system 10 includes a cassette (shown in FIG. 1 as cassette 12), the rigid tube 12T is located downstream of the cassette and is connected to or integrated with an outlet tube (shown in FIG. 1 as outlet tube 6). If the system does not include a cassette, the tube 12T can be located downstream of whatever pumping mechanism (shown in FIG. 1 as pumping mechanism 15) exists. In the case of gravity flow where no pumping mechanism exists other than gravity, the tube 12T can be located between the source (shown in FIG. 1 as source 2) and the patient (shown in FIG. 1 as patient 8), downstream of a conventional clamp or valve (not shown) for controlling the flow of the infusion fluid 30. The infusion fluid 30 enters the tube 12T through an inlet and flows to an outlet. The upstream transducer 16T is coupled to the tube 12T adjacent to the inlet by a coupling element 16C. The downstream transducer 18T is coupled to the tube 12T adjacent to the outlet by a coupling element 18C.

Taking as an example the embodiment in FIG. 2D, FIG. 3 shows one embodiment of the flow sensor circuits which may be utilized to monitor the flow rate of the infusion fluid 30. In this case, the transducers 16T, 18T can be transducers or can each include separate emitters or transmitters 16E, 18E and receivers 16R, 18R. The transmitter and receiver function can be accomplished by the same transducer or by separate transducers performing each a single function. The transmitters 16E and 18E can emit ultrasonic signals and the receivers 16R and 18R receive the ultrasonic signals from the transmitters 18E and 16E. The receivers 16R and 18R can include noise cancelling components such as noise cancelling microphones 16M and 18M. The microcontroller 302 (also referred to as a hardware processor herein which could include one or more hardware processors) commands a frequency generator 304 to generate a voltage signal with the programmed frequency f. A driver 306 amplifies the signal and sends it to the emitter 16E of one of the transducers 16T. The emitter 16E generates an ultrasonic signal S that propagates through the fluid 30 and is detected by the receiver 18R at the other end of the tube, which generates a voltage signal proportional to the received ultrasonic signal R. The emitted signal S and the received signal R are fed to a phase comparator 308 that generates a signal proportional to the phase difference between the two signals. A signal conditioning stage 310 amplifies and filters the signal and sends it to an analog to digital converter 312 that converts it to a digital phase signal received by the microcontroller 302. The microcontroller 302 controls the switches 314A, 314B, 314C, 314D that select the propagation direction of the ultrasonic signal. In this embodiment the signal S is emitted alternatively with and against the flow direction. When the signal S is to be emitted with the direction of fluid flow, the normally closed switches 314A and 314B are closed and the normally open switches 314C and 314D are open; and when the signal S is to be emitted in the opposite direction, against the direction of fluid flow, the microcontroller 302 opens switches 314A and 314B and closes switches 314C and 314D. The transmitter and receiver transducers can be piezoelectric, electromagnetic, or microelectromechanical systems (MEMS) based in construction.

Taking as an example the embodiment of FIG. 20, FIG. 3A shows another embodiment of the flow sensor circuits which may be utilized to monitor the flow rate of the infusion fluid 30. In this case the transducers 16T and 18T still contain separate emitters 16E and 18E and receivers 16R and 18R, but the transducers 16T and 18T are operated continuously. In other words, the case of simultaneous transmission with separate transmitter and receiver transducers is illustrated. There are two separate channels, one for the acoustic signal propagating against the flow and one for the acoustic signal propagating with the flow. The programmed frequencies f₁ and f₂ of the signals S1 and S2 in the two channels are different, so they can be separated by the phase comparators 308A and 3088. The microcontroller 302 commands a frequency generator 304A to generate a voltage signal with the programmed frequency f₁. A driver 306A amplifies the signal S1 and sends it to the emitter 16E of one of the transducers 16T. The emitter 16E generates an ultrasonic signal that propagates through the fluid 30 and is detected by the receiver 18R at the other end of the tube, which generates a voltage signal proportional to the received ultrasonic signal R1. The emitted signal S1 and the received signal R1 are fed to a phase comparator 308A that generates a signal proportional to the phase difference between the two signals. A signal conditioning stage 31 OA amplifies and filters the signal and sends it to an analog to digital converter 312A that converts it to a digital phase signal received by the microcontroller 302. The microcontroller 302 further commands a frequency generator 3048 to generate a voltage signal with the programmed frequency f₂. A driver 3068 amplifies the signal S2 and sends it to the emitter 18E of one of the transducers 18T. The emitter 18E generates an ultrasonic signal that propagates through the fluid 30 and is detected by the receiver 16R at the other end of the tube, which generates a voltage signal proportional to the received ultrasonic signal R2. The emitted signal S2 and the received signal R2 are fed to a phase comparator 3088 that generates a signal proportional to the phase difference between the two signals. A signal conditioning stage 31 OB amplifies and filters the signal and sends it to an analog to digital converter 3128 that converts it to a digital phase signal received by the microcontroller 302. The transmitter and receiver transducers can be piezoelectric, electromagnetic, or microelectromechanical systems (MEMS) based in construction.

Again taking as an example the embodiment of FIG. 2B, FIG. 3B shows another embodiment of the flow sensor circuits which may be utilized to monitor the flow rate of the infusion fluid 30. In this case, the transducers 16T and 18T perform both functions of emitting and receiving the ultrasonic signals. The transducers 16T and 18T transmit simultaneously and are operated continuously. There are still two separate channels. The frequencies f1, f2 of the signals S1 and S2 in the two channels are different, so they can be separated by the phase comparators 308A and 308B. The voltage at the terminals of each of the transducers 16T and 18T is the sum of the emitted signals S1 and S2 voltages from the drivers 306A and 306B and the voltages generated by the transducers 16T and 18T, which is proportional to the received signals R2 and R1. Difference amplifiers 316A and 316B subtract the emitted signals S1 or S2 from this sum, outputting the received signals R2 or R1 respectively. The microcontroller 302 (also referred to as a processor herein which could include one or more processors) commands frequency generator 304A to generate a voltage signal with the programmed frequency f₁. A driver 306A amplifies the signal S1 and sends it to the transducer 16T. The transducer 16T generates an ultrasonic signal that propagates through the fluid 30 and is detected by the transducer 18T at the other end of the tube, which generates a voltage signal of the sum of the received ultrasonic signal R1 and the signal S2 transmitted by the transducer 18T. Difference amplifier 316A subtracts the emitted signal S2 from this sum, outputting the received signal R1. The emitted signal S1 and the received signal R1 are fed to a phase comparator 308A that generates a signal proportional to the phase difference between the two signals. A signal conditioning stage 310A amplifies and filters the signal and sends it to an analog to digital converter 312A that converts it to a digital phase signal received by the microcontroller 302. The microcontroller 302 further commands frequency generator 304B to generate a voltage signal with the programmed frequency f₂. A driver 306B amplifies the signal S2 and sends it to the transducer 18T. The transducer 18T generates an ultrasonic signal that propagates through the fluid 30 and is detected by the transducer 16T at the other end of the tube, which generates a voltage signal of the sum of the received ultrasonic signal R2 and the signal S1 transmitted by the transducer 16T. Difference amplifier 316B subtracts the emitted signal S1 from this sum, outputting the received signal R2. The emitted signal S2 and the received signal R2 are fed to a phase comparator 308B that generates a signal proportional to the phase difference between the two signals. A signal conditioning stage 310B amplifies and filters the signal and sends it to an analog to digital converter 312B that converts it to a digital phase signal received by the microcontroller 302. The transmitter and receiver transducers can be piezoelectric, electromagnetic, or microelectromechanical systems (MEMS) based in construction.

As shown in FIG. 1 (and as further detailed in other embodiments herein using the same or different reference numbers), the hardware processor 20 is in electronic communication with the infusion pump 11, the upstream acoustic sensor 16, the downstream acoustic sensor 18, the one memory 22, the proximal air-in-line sensor 24, the proximal pressure sensor 25, the distal pressure sensor 26, and the distal air-in-line air sensor 28. The memory 22 contains the programming code 23 which is configured to be executed by the processor 20. The programming code 23 is configured to determine the volumetric flow rate of the infusion fluid 30 along the flow path 14 based on the upstream acoustic signal detected by the downstream acoustic sensor 18 and on the downstream acoustic signal detected by the upstream acoustic sensor 16, and to automatically adjust the infusion pump 11, based on the determined volumetric flow rate, to achieve a desired volumetric flow rate of the infusion fluid 30 along the flow path 14.

In the embodiments of FIGS. 3, 3A and 3B, the hardware processor 20 executing the programming code 23 (shown in FIG. 1) can determine the volumetric flow rate of the infusion fluid 30 along the flow path 14 based on a first phase delay of the upstream acoustic signal between the one upstream acoustic sensor 16 and the downstream acoustic sensor 18, or on a second phase delay of the downstream acoustic signal between the downstream acoustic sensor 18 and the upstream acoustic sensor 16.

In the embodiment of FIG. 1, the hardware processor 20 executing the programming code 23 is configured to determine the volumetric flow rate of the infusion fluid 30 along the flow path 14 by using the algorithm Q=V*A. In the algorithm, Q includes the volumetric flow rate, V includes a velocity of the infusion fluid 30 generated by the infusion pump 11, and A includes a cross-sectional area of the flow path 14. Moreover, V=(L/2)*(1/t₁−1/t₂), wherein L includes a length between the upstream location 14a and the downstream location 14b, t₁ includes a first time it takes the upstream acoustic signal to travel from the one upstream acoustic sensor 16 to the downstream acoustic sensor 18, and t₂ includes a second time it takes the downstream acoustic signal to travel from the downstream acoustic sensor 18 to the one upstream acoustic sensor 16. In other words, t₁−t₂ can be thought of as the transit time of the signal or delta t. The distance between the sensors divided by delta t is the speed of the flow stream. The speed times the area of flow path equals the volumetric flow rate.

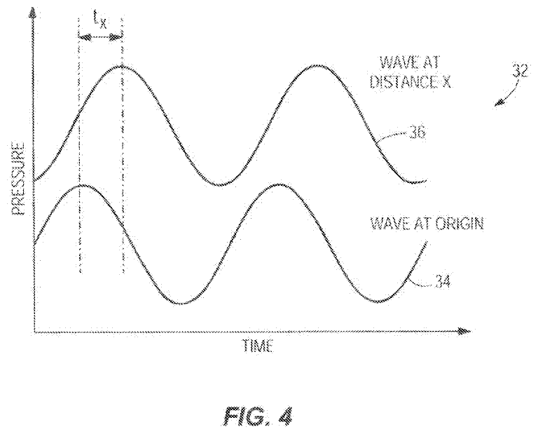

FIG. 4 illustrates a graph 32 of one embodiment of two acoustic pressure function curves 34 and 36 plotting time on the X-axis and pressure on the Y-axis to show the phase delay between the pressure function curves 34 and 36. Acoustic pressure function curve 34 includes the pressure wave at the origin of the upstream acoustic sensor 16 or 16T of FIG. 28. Acoustic pressure function curve 36 includes the pressure wave at distance x from the origin of the downstream acoustic sensor 18 or 18T of FIG. 2B. There is a delay time tₓ between the two acoustic pressure function curves 34 and 36. The two acoustic pressure function curves 34 and 36 represent periodic variations in time and space of the pressure in the liquid. It can be assumed that the pressure at the source is a simple sinusoidal function represented by P(0,t)=P₀ sin(2*r*/*t), where P₀ includes pressure amplitude, f includes frequency of the sound wave, and t includes time.

At the distance x, the wave 36 is delayed by the time tₓ or, to express it in another way, the argument of the sine function is changed by the phase angle φ so that

P(x,t)=P₀ sin [2*π*f*(t−tₓ)]=P₀ sin(2*π*f*t+φ). Using an electronic circuit called a phase discriminator, which is known in the art, to measure the phase angle between the two waves, the delay time between the wave 34 emitted and the wave 36 received at a certain distance may be calculated as being tₓ=−φI 2*π*f. In order to reduce the size of the measurement apparatus, shorter time measurements may be required due to the short distance between the transmitter and receiver. These shorter time measurements over the shorter distances can be accomplished through the use of the phase discriminator. In order to increase the resolution of the fluid flow without restricting the area of the flow channel a differential measurement is used. This measurement is done by determining the phase delay between the transmitter and receiver of a continuous signal. To convert this phase angle measurement to fluid speed it is necessary to take the period of the repetitive signal times the angle, according to the tₓ equation shown above.

For example, in the case of a short distance between the transmitter and receiver (less than a full cycle), given a predetermined/designed oscillator frequency f=5 MHz and a measured phase delay angle of 12 degrees, the method might determine:

Calculated time delay tₓ=−φI2*π*f

$$tx = - 12 deg . / ( 2 π 5 MHz ) = 42 nsecs$$

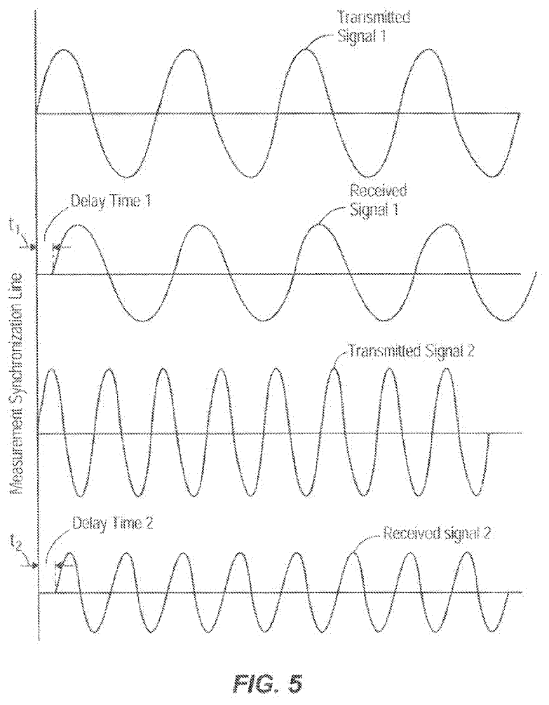

Thus, this method allows a small time delay to be measured. Utilization of signal phase shift allows measurement of a very small time delay. Advantageously, this can translate into accurate measurements over very short distances too. This is accomplished by utilizing a carrier signal phase shift between the signal emitted by the transmitter source to the signal at the receiver. The process described herein can be further illustrated in FIGS. 4 and 5.

The determined time delay can be used to determine the velocity V of the fluid, which in turn can be used to determine the volumetric flow rate Q. An illustration of velocity computation follows with a hypothetical numerical example where: V=(L)*(1/t₁−1/t₂); tx=(1/t₁−1/t₂); and L=distance between transmitter and receiver.

In a phase approach, over longer distances than the example above, several full cycles may exist between the transmitter and the receiver due to the distance or length L between them. For purposes of illustration below we will utilize 50 full cycles, plus a partial cycle that is measured as a phase delay. Assuming L is predetermined or designed to equal. 0.005 m, the frequency of the carrier is 10 kHz, and ϕ=143 deg (measured):

Given the above tₓ will be:

(converting the angle to time)

$$tx = ( 143 deg + 50 * 360 deg ) / 2 * π * 10 kHz = 835 μ S$$

(then converting the time to velocity)

$$V = \frac{\text{.005} m}{tx} = 5.98 m / s$$

Area, A, is the cross sectional area of the flow path (in this example the flow path is a tube or tubing with an inside diameter of 0.0003 meters)

$$Area = {( \frac{\text{.0003} m}{2} )}^{2} * π = 0.071 {mm}^{2}$$

This gives the volumetric flow rate Q=V*A

$$Q = V * Area = 1523 ml / hr$$

In the above equation, Q includes the volumetric flow rate, V includes a velocity of the infusion fluid 30 generated by the infusion pump 11, and A includes a cross-sectional area of the flow path 14.

So to review and summarize, the overall time propagation of the sound waves 34 and 36 will be affected by the flow of the fluid through the tubing and/or channel. There will be a difference in the delays since the propagation occurs faster downstream than upstream so that t₁=L/C+V, and t₂=L/C−V, wherein t₁ includes the time of sound propagation downstream, t₂ includes the time of sound propagation upstream, L includes a length of sound propagation path in the fluid channel, C includes sound velocity in the fluid, and v includes a velocity of fluid generated by the infuser. If the times are known, the following equation can be used 1/t₁−1/t₂=(2*V)/L. From this equation, the following equation can be obtained V=(L/2)*(1/t₁−1/t₂). If the cross section area of the flow path A is known, the volumetric flow rate Q can be calculated using the equation Q=V*A. In the embodiment of FIG. 1, the hardware processor 20 executing the programming code 23 can determine the volumetric flow rate of the infusion fluid 30 along the flow path 14 based on the phase delay by using the equations above.

FIG. 5 illustrates one embodiment of a set of graphs in which two different frequencies are transmitted, received, and processed by two different transducers respectively. The graphs depict the varying phase delays of the differing frequencies.

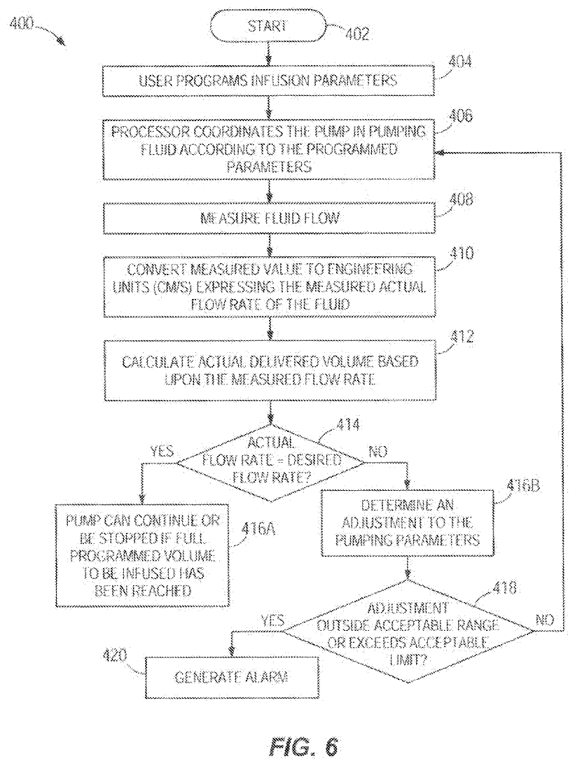

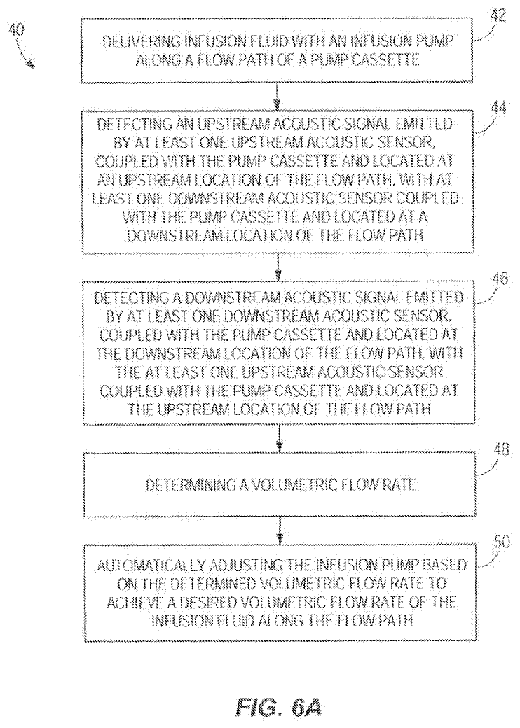

FIG. 6 illustrates a flowchart of one embodiment of a method 400 of automatically detecting and adjusting a volumetric flow rate delivered by an infusion pump. FIG. 6 can also be thought of as a block diagram of closed loop control using flow sensing transduces. The method 400 may utilize any of the infusion systems disclosed herein. In other embodiments, the method 400 may utilize varying infusion systems. The method can start at node 402. In step 404, the infusion parameters including desired fluid flow rate, duration and dose are programmed by the user. Initially prompted by a start command from a user, in step 406, the hardware processor coordinates the pump in pumping fluid according to the programmed parameters. In step 408, the fluid flow is measured as described above and further explained with reference to FIG. 6A below. In step 410, the measured value is converted to engineering units (cm/s, m/s, etc.) expressing the measured actual flow rate of the fluid. Scaling based on the physical dimensions of the tube or flow channel and time offsets may be utilized. In step 412, the actual delivered volume is calculated based upon the measured flow rate and in step 414, the actual delivered volume is compared to the programmed or desired volume based upon the programmed delivery flow rate. If the actual flow rate is equal to the desired flow rate and thus the delivered volume is correct, then the method proceeds to step 416A and the pump can continue as is or be stopped if the full programmed volume to be infused has been reached. If the actual flow deviates or is not equal to the desired flow and thus the delivered volume is incorrect, the method proceeds to step 4168 where an adjustment to the pumping parameters such as the programmed or desire flow rate is determined. In optional step 418 the adjustment or the new program parameter can be evaluated for acceptability by a processor against a limit or range of limits in a memory. The limits can be hard coded into the pump or included in a user-customizable drug library that can be downloaded to the processor or memory of the pump over a network. If the adjustment or adjusted program parameter is outside the acceptable range or exceeds an acceptable limit, then an optional alarm is generated in step 420. The alarm can be communicated visually, audibly, by other perceptible means or merely relayed electronically to a remote device. If the adjustment or adjusted program parameter such as a new desired flow rate is within the acceptable range or limit, the method moves back to step 406 and the method continues with the pump processor being automatically programmed to pump the fluid according to the newly adjusted program parameter such as a new flow rate. In other embodiments, one or more steps of the method 400 may be changed in substance or order, one or more steps of the method 400 may not be followed, or one or more additional steps may be added.

With reference to FIG. 6A, in one embodiment the flow measurement step 408 and others from FIG. 6 are disclosed in greater detail as the steps of a flow measurement and automatic adjustment process 40. In step 42, infusion fluid is delivered with an infusion pump along a flow path of a pump cassette. In step 44, an upstream acoustic signal emitted by the upstream acoustic sensor, coupled with the pump cassette and located at an upstream location of the flow path, is detected with the downstream acoustic sensor coupled with the pump cassette and located at a downstream location of the flow path. The upstream acoustic sensor may include at least one ultrasonic upstream acoustic sensor. In other embodiments, the upstream acoustic sensor may vary. In one embodiment, step 44 may include detecting the upstream acoustic signal emitted by the upstream acoustic sensor by receiving the upstream acoustic signal with a first noise cancelling component such as a first noise cancelling microphone.

In step 46, a downstream acoustic signal emitted by the downstream acoustic sensor, coupled with the pump cassette and located at the downstream location of the flow path, is detected with the upstream acoustic sensor coupled with the pump cassette and located at the upstream location of the flow path. The downstream acoustic sensor may include one or more ultrasonic upstream acoustic sensor. In other embodiments, the downstream acoustic sensor may vary. In one embodiment, step 46 may include detecting the downstream acoustic signal emitted by the downstream acoustic sensor by receiving the downstream acoustic signal with a second noise cancelling component such as a second noise cancelling microphone. In step 48, a volumetric flow rate is determined, with the hardware processor, of the infusion fluid along the flow path based on the upstream acoustic signal detected by the downstream acoustic sensor and the downstream acoustic signal detected by the upstream acoustic sensor.

In one embodiment, step 48 may include determining, with the hardware processor, the volumetric flow rate of the infusion fluid along the flow path by determining a first phase delay of the upstream acoustic signal between the upstream acoustic sensor and the downstream acoustic sensor, or by determining a second phase delay of the downstream acoustic signal between the downstream acoustic sensor and the upstream acoustic sensor. This may be done using an algorithm executed by the hardware processor 20.

In another embodiment, step 48 may include determining, with the processor, the volumetric flow rate of the infusion fluid along the flow path by using the algorithm Q=V*A, wherein Q includes the volumetric flow rate, V includes a velocity of the infusion fluid generated by the infusion pump, A includes a cross-section area of the flow path, and V=(L/2)*(1/t₁−1/t₂), wherein L includes a length between the upstream location and the downstream location, t₁ includes a first time it takes the upstream acoustic signal to travel from the upstream acoustic sensor to the downstream acoustic sensor, and t₂ includes a second time it takes the downstream acoustic signal to travel from the downstream acoustic sensor to the upstream acoustic sensor.

In step 50, the infusion pump is automatically adjusted, with the hardware processor, based on the determined volumetric flow rate to achieve a desired volumetric flow rate of the infusion fluid along the flow path. In other embodiments, one or more steps of the method 40 may be changed in substance or order, one or more steps of the method 40 may not be followed, or one or more additional steps may be added. It is noted that the method 40 may utilize any of the system or method embodiments disclosed herein. One or more embodiments of the disclosure allows for improved accuracy of determining how much infusion fluid is being delivered to the patient while decreasing manufacturing cost of the infusion system. It should be understood, of course, that the foregoing relates to exemplary embodiments of the disclosure and that modifications may be made without departing from the scope of the disclosure as set forth in the following claims.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

Reference to any prior art in this specification is not, and should not be taken as, an acknowledgement or any form of suggestion that that prior art forms part of the common general knowledge in the field of endeavour in any country in the world.

The disclosed apparatus and systems may also be said broadly to consist in the parts, elements and features referred to or indicated in the specification of the application, individually or collectively, in any or all combinations of two or more of said parts, elements or features.

Where, in the foregoing description reference has been made to integers or components having known equivalents thereof, those integers are herein incorporated as if individually set forth.

Depending on the embodiment, certain acts, events, or functions of any of the algorithms, methods, or processes described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially.

It should be noted that various changes and modifications to the presently preferred embodiments described herein will be apparent to those skilled in the art. Such changes and modifications may be made without departing from the spirit and scope of the disclosed apparatus and systems and without diminishing its attendant advantages. For instance, various components may be repositioned as desired. It is therefore intended that such changes and modifications be included within the scope of the disclosed apparatus and systems. Moreover, not all of the features, aspects and advantages are necessarily required to practice the disclosed apparatus and systems. Accordingly, the scope of the disclosed apparatus and systems is intended to be defined only by the claims that follow.

1.-37. (canceled)

38. An infusion system comprising:
an infusion pump configured to pump an infusion fluid along a flow path;
a first acoustic sensor positioned at a first location along the flow path, the first acoustic sensor configured to detect a first acoustic signal; and
a second acoustic sensor positioned at a second location downstream from the first acoustic sensor along the flow path, the second acoustic sensor configured to detect a second acoustic signal.

39. The infusion system of claim 38, wherein the first acoustic signal originated from the second acoustic sensor and the second acoustic signal originated from the first acoustic sensor.

40. The infusion system of claim 38, wherein the first acoustic sensor comprises a first transducer and the second acoustic sensor comprises a second transducer.

41. The infusion system of claim 38, wherein the first acoustic sensor comprises a first transmitter and a first receiver and the second acoustic sensor comprises a second transmitter and a second receiver.

42. The infusion system of claim 41, wherein the first receiver and the second receiver each comprise at least one noise cancelling component.

43. The infusion system of claim 38, further comprising a controller configured to: determine a first volumetric flow rate of the infusion fluid based on the detected first acoustic signal and the detected second acoustic signal, and control the infusion pump to pump the infusion fluid at a second volumetric flow rate based on the detected first volumetric flow rate.

44. The infusion system of claim 43, wherein the first volumetric flow rate of the infusion fluid is calculated over each stroke of the infusion pump.

45. The infusion system of claim 43, wherein the first volumetric flow rate is determined based on a first phase delay associated the first acoustic signal.

46. The infusion system of claim 43, wherein the first volumetric flow rate is determined based on a second phase delay associated the second acoustic signal.

47. The infusion system of claim 43, wherein the first volumetric flow rate is determined based on a length between the first location and the second location.

48. The infusion system of claim 43, wherein the first volumetric flow rate is determined based on a first time it takes the first acoustic signal to travel between the second acoustic sensor and the first acoustic sensor.

49. The infusion system of claim 43, wherein the first volumetric flow rate is determined based on a first time it takes the second acoustic signal to travel between the first acoustic sensor and the second acoustic sensor.

50. A method of determining a volumetric flow rate of an infusion fluid along a flow path in an infusion pump, the method comprising:
detecting a first acoustic signal from a first acoustic sensor positioned at a first location along the flow path;
detecting a second acoustic signal from a second acoustic sensor positioned at a second location downstream from the first acoustic sensor along the flow path; and
determining a first volumetric flow rate of the infusion fluid based on the detected first acoustic signal and the detected second acoustic signal.

51. The method of claim 50, wherein the first acoustic signal originated from the second acoustic sensor and the second acoustic signal originated from the first acoustic sensor.

52. The method of claim 50, wherein the first acoustic sensor comprises a first transducer and the second acoustic sensor comprises a second transducer.

53. The method of claim 50, wherein the first acoustic sensor comprises a first transmitter and a first receiver and the second acoustic sensor comprises a second transmitter and a second receiver.

54. The method of claim 50, wherein the first volumetric flow rate of the infusion fluid is calculated over each stroke of the infusion pump.

55. The method of claim 50, wherein the first volumetric flow rate is determined based on a first phase delay associated the first acoustic signal.

56. The method of claim 50, wherein the first volumetric flow rate is determined based on a second phase delay associated the second acoustic signal.

57. The method of claim 50, wherein the first volumetric flow rate is determined based on a length between the first location and the second location.
